(19) United States
(12) Patent Application Publication
(10) Pub. No.: US 2012/0130467 A1
(43) Pub. Date: May 24, 2012

(54) INTERFACIAL STENT AND METHOD OF MAINTAINING PATENCY OF SURGICAL FENESTRATIONS

(76) Inventors: Nathan R. Selden, Portland, OR (US); Rachel Dreilinger, Lake Oswego, OR (US)

(21) Appl. No.: 13/386,884

(22) PCT Filed: Jul. 26, 2010
(86) PCT No.: PCT/US10/43253

§ 371 Date: Jan. 24, 2012

Related U.S. Application Data
(60) Provisional application No. 61/228,510, filed on Jul. 24, 2009.

(51) Int. Cl. A61F 2/82

(52) U.S. Cl. 623/1.2

(57) ABSTRACT

A method according to one embodiment for maintaining patency of an opening inside the human body comprises introducing a radially self-expanding hollow stent into the opening while the stent is retained in a radially compressed state, wherein the stent has enlarged ends and a reduced intermediate portion. The stent is introduced into the opening such that its intermediate portion extends through the opening and the enlarged ends are positioned outside of the opening. Once deployed, at least the end portions of the stent expand on opposing faces of the opening to resist dislodgement of the stent from the opening. The stent is preferably biodegradable, such that it is eliminated from the surgical site over a period of weeks to months, by which time the patency of the opening is more assured. The method can be used in combination with, for example, an endoscopic surgical method such as endoscopic third ventriculostomy for treating hydrocephalus of a brain.

### CROSS-REFERENCE TO RELATED APPLICATION

The present application claims the benefit of U.S. Provisional Application No. 61/228,510, filed Jul. 24, 2009, which is incorporated herein by reference.

### FIELD

The present disclosure relates to implantable stents used inside the human body for medical purposes.

### BACKGROUND

The human body includes many anatomical pathways through which body fluids, such as blood or cerebrospinal fluid (CSF), must pass to maintain proper biological function. Examples of such pathways are elongated blood vessels (such as the coronary arteries) and other extended passageways that define a lumen (such as the aqueduct of Sylvius in the ventricular system of the brain). Obstructions of biological lumens can cause serious medical problems, such as tissue ischemia secondary to occlusion of an artery, or hydrocephalus caused by disruption of the flow of CSF through the ventricular system.

In the case of obstruction of an elongated vessel, such as stenosis of a blood vessel in the cardiovascular system, implantable intra-luminal stents have been used to maintain patency of the vessel lumen. Intravascular stents are commonly placed in an atherosclerotic coronary artery to reestablish perfusion to ischemic cardiac tissue. Coronary stents are introduced along a catheter to a site of occlusion during an angioplasty procedure. The stents, which are typically tubular in shape, may be expanded mechanically or by the introduction of pressurized air into a balloon placed in the lumen of the stent. Coronary stents are not usually designed to be biodegradable, because they are intended to provide long-term mechanical support to maintain patency of the vessel lumen.

In addition to using stents, surgeons often employ other operative techniques to reestablish normal flow of fluids through biological pathways in the body. For example, an artificial opening such as a surgical fenestration may be created in a biological interface (such as a membrane or other tissue barrier) to either reopen a natural pathway or to create a new pathway for therapeutic purposes. Endoscopic surgery procedures may involve fenestration of a biological interface inside the body, in which a small opening is surgically created to establish or facilitate communication of such fluids as blood, bile, aqueous humor or cerebrospinal fluid (CSF).

Endoscopic third ventriculostomy or endoscopic third ventriculocistemostomy (ETV) is an example of a particular endoscopic procedure performed to treat pathological disruption of normal biological fluid flow. ETV is a procedure used for relieving hydrocephalus, a medical condition in which cerebrospinal fluid (CSF) accumulates in the ventricles of the brain due to obstruction of the flow of CSF within or from the ventricles. The accumulation of CSF increases pressure inside the brain, which in turn causes enlargement of the cranium and compression of intracranial brain tissue. Hydrocephalus most frequently occurs in young children, but is also found among adults, and is usually accompanied by neurological deterioration or death.

A standard method to relieve hydrocephalus is to shunt CSF from the brain into the abdominal, venous or peritoneal space. The shunt procedure employs a valved CSF shunt system connected to a plastic drainage line that diverts CSF out of the brain. A specific example of this procedure is ventriculoperitoneal (VP) drainage, which is commonly used to treat hydrocephalus. However, such shunts often fail when they become infected or require surgical revision to relieve obstruction of the shunt. To help avoid such problems, endoscopic third ventriculostomy (ETV) is now commonly used to treat obstructive hydrocephalus, such as that caused by an obstruction of the Aqueduct of Sylvius that communicates between the third and fourth ventricles. ETV creates a surgical fenestration between the third ventricle and the subarachnoid space to permit drainage of excess CSF.

ETV can be performed by placing a burr-hole anterior to the coronal suture of the skull and introducing an endoscope through the brain, into the lateral ventricle and through the foramen of Monro to gain access to the floor of the third ventricle. A fenestration (a ventriculostomy opening) is then surgically created in the floor of the third ventricle, anterior to the basilar artery. The fenestration can be made, for example, by introducing through the floor of the ventricle a blunt guide wire, closed forceps, laser, ultrasonic probe, or the tip of the endoscope itself. The fenestration hole is then enlarged to approximately 5 mm by expanding the tip of a Fogarty balloon catheter in the fenestration or by using an instrument designed for purposeful dilation of the fenestration. One advantage of the ETV procedure is that it does not require an indwelling, permanent shunt catheter that is subject to occlusion or infection.

Although ETV has greatly improved the treatment of hydrocephalus, the ventriculostomy opening sometimes becomes partially or completely occluded as scar tissue forms at the fenestration site. Even in carefully selected patients with obstructive hydrocephalus, technically successful endoscopic third ventriculostomy results in alleviation of hydrocephalus in 60% to 70% of subjects, with up to 40% of subjects having an unsatisfactory clinical outcome. A significant proportion of patients who fail to respond to ETV suffer from secondary closure of the ETV site due to scarring and/or arachnoidal adhesions, and may require subsequent surgical procedures to reestablish patency of the opening or alternatively may result in lifetime ventricular shunt dependency. This problem with ETV illustrates a more general problem with many endoscopic and other surgical procedures that create artificial openings inside the human body. Surgically created openings in biological interfaces, such as the walls of an organ or other anatomic structures, frequently close as a result of a normal inflammation and healing processes. It would therefore be useful to have a method or device that would maintain the patency of such openings for a sustained period of time.

### SUMMARY

The present disclosure provides a method for maintaining patency of an opening through an interface inside the human body by introducing a radially self-expanding hollow stent into the opening utilizing a delivery device that retains the stent in a radially compressed state as it is introduced into the body. The stent has enlarged ends and a constricted intermediate portion. The shape of the stent allows it to be placed with its constricted intermediate portion situated in the opening while the enlarged ends remain outside of the opening on opposite sides of the opening. The self-expanding stent is allowed to expand in situ such that the enlarged ends inhibit dislodgement of the stent from the opening. A lumen through the stent permits the free flow of fluid through the opening while maintaining patency of the opening.

In particular embodiments, the stent is biodegradable, such that it degrades or otherwise dissolves over time (for example in one to six months). Once the stent has degraded after this period of time, the incidence of scarring or other closure of the opening is reduced. The method can be implemented using an endoscopic surgical procedure for treating hydrocephalus of the brain that increases the success rate of the surgery and reduces the chance of secondary failure. Such a method can include introducing an endoscope into the third ventricle of the brain; fenestrating the floor of the third ventricle to create an opening that fluidly communicates between the third ventricle and subarachnoid space; enlarging the opening; and placing the stent into the opening.

Also disclosed herein is an interfacial stent for maintaining patency of an opening in a biological interface (such as a wall of an organ or substructure thereof, such as a ventricle of a brain) in a human body. The stent includes two enlarged ends and a constricted intermediate portion. The stent is self-expandable, for example being made of a material that has resilient memory, and may be biodegradable. In particular examples, when the two enlarged ends are expanded, each has a diameter substantially greater than a diameter of the constricted intermediate portion that extends through and fills the opening, and/or about the same or greater than the length of the stent.

In a representative embodiment, a stent comprises a non-expandable intermediate portion defining a lumen and having an outer diameter. The stent further comprises first and second self-expandable end portions coupled to opposite ends of the intermediate portion. Each of the first and second end portions comprise a plurality of circumferentially arrayed, axially elongated fingers that are radially expandable between a compressed state for delivery into a patient and an expanded state for deployment in the patient. Each of the end portions desirably has a maximum diameter in their expanded state that is greater than the outer diameter of the intermediate portion.

In another representative embodiment, a method for maintaining patency of an opening inside the human body comprises introducing a radially self-expanding stent into the body in a compressed state. The stent comprises a non-expandable intermediate portion defining a lumen and having an outer diameter. The stent further comprises first and second self-expandable end portions coupled to opposite ends of the intermediate portion. Each of the first and second end portions comprise a plurality of circumferentially arrayed, axially elongated fingers. After the stent is introduced into the body, the first end portion is positioned on one side of the opening and the fingers of the first end portion are allowed to radially expand to an expanded state. The second end portion of the stent is positioned on an opposite side of the opening and the fingers of the second end portion are allowed to radially expand to an expanded state such that the expanded fingers retain the stent within the opening.

Other features and advantages of the invention will become more readily understandable from the following detailed description and figures.

### BRIEF DESCRIPTION OF THE DRAWINGS

FIG. 1 is a schematic sagittal section view of the human brain in a child.

FIG. 2 is a schematic top view of a portion of the floor of the third ventricle in the human brain, illustrating a surgical fenestration in the floor of the ventricle.

FIG. 3 is a cross-sectional view of the floor of the third ventricle showing a surgical fenestration.

FIGS. 4A and 4B are perspective and side views, respectively, of a stent, according to one embodiment, that can be used to maintain the patency of surgical fenestration, with the stent shown in an expanded state.

FIGS. 5A and 5B are perspective and side views, respectively, of the stent of FIG. 4 shown in a radially compressed state.

FIG. 6 is a perspective view of a delivery apparatus that can be used to implant a stent, such as the stent shown in FIG. 4, in a surgical fenestration.

FIGS. 7-20 illustrate the delivery and implantation of the stent of FIG. 4 in a surgical fenestration using the delivery apparatus shown in FIG. 6.

FIGS. 21-22 are perspective views of another embodiment of a stent shown in a compressed state and an expanded state, respectively.

### DETAILED DESCRIPTION

### A. Terms

In the present description, the terms “opening”, “hole”, “orifice”, “fenestration”, “perforation” and “stoma” all refer to an opening, either naturally existing or artificially created, through an interface of a human body part such as a tissue or membrane. Such interfaces may be found either externally (for example through an ear lobe or other skin surface) or internally (such as the wall of a hollow organ, or wall of a substructure of an organ, such as the ventricles of the brain or the interventricular lumen). In contrast, the term “lumen” refers to the open space within an elongated tubular vessel. Hence an opening, hole, fenestration or perforation is typically present in tissue interface, in contrast to a lumen, which extends through a tubular or elongated extended tissue structure. In addition, the embodiments of the stent disclosed herein are devised to maintain the patency of an artificially created opening, instead of restoring patency of a pre-existing lumen that has become occluded by a pathological process (such as atherosclerosis).

### B. Disclosed Embodiments

The disclosed embodiments of the stent are generally designed to maintain patency of an anatomic interface opening (such as a surgically created fenestration) along the entire length of the opening, by retention of the stent within the interface opening by its enlarged ends disposed on opposing faces of the opening. This is distinguished from the prior art use of a stent in a lumen of an elongated tubular vessel such as vascular artery, in which the stent occupies only an intermediate section of an elongated vessel and is contained entirely within the lumen of the elongated vessel.

Several representative embodiments of the stent and methods of its use are disclosed herein for purposes of illustrating how to make and use certain examples of the invention. The representative embodiments are not intended to be limiting in any way. Further embodiments are disclosed in co-pending U.S. Patent Application Publication No. 2007/0179426 (U.S. application Ser. No. 11/596,270), which is incorporated herein by reference.

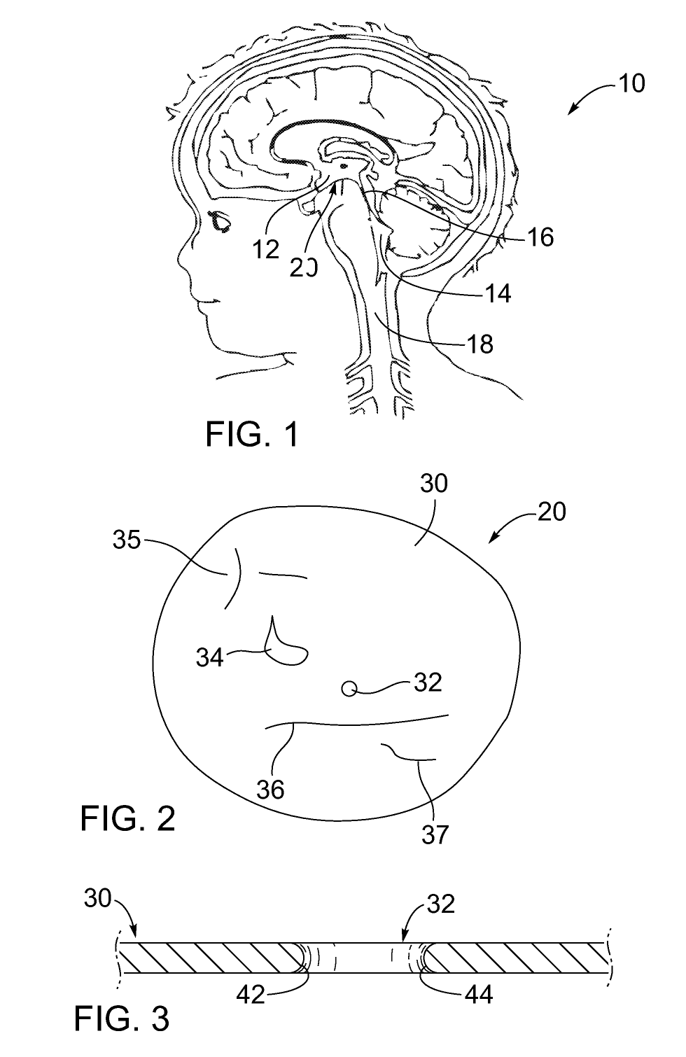

FIG. 1 shows a schematic sagittal section view of human brain 10. Viewable from the sagittal section view is a third ventricle 12, a fourth ventricle 14, and an Aqueduct of Sylvius 16 which in a normal condition communicates between third ventricle 12 and fourth ventricle 14. CSF from fourth ventricle 14 circulates around spinal cord 18 which depends from the brainstem. Also shown in this view is the floor of the third ventricle 20.

A subject who suffers obstructive hydrocephalus often has a blockage of the normal flow of CSF through the ventricular system and the subarachnoid space. For example, a barrier to flow can form within an obstructed Aqueduct of Sylvius 16, which allows abnormal amounts of CSF to accumulate in the proximal portions of the ventricular system, for example in the third ventricle 12 and lateral ventricles. This CSF accumulation is a common cause of hydrocephalus which ultimately causes megalocephaly (enlargement of the head), and compression of neural pathways that leads to deterioration of neurological status, disability and/or death.

FIG. 2 is a schematic top view of wall 30 of the floor of the third ventricle 20 in human brain 10. This view schematically represents what is visible through an endoscope (not shown in FIG. 2) that is introduced into the third ventricle 12 through an endoscopic channel that communicates with a surgical opening through the skull anterior to the coronal suture (not shown). Schematically shown in FIG. 2 are several parts in the brain visible through the semi-transparent floor of the third ventricle 20, including hypophyseal portal veins 35, pituitary gland 34, posterior cerebral artery 36, and posterior perforating arteries 37.

During endoscopic third ventriculostomy (ETV), a fenestration 32 is created in the floor of third ventricle 20 to re-establish flow of cerebrospinal fluid from the third ventricle 12 (FIG. 1) to the subarachnoid space (not shown) underneath the floor of the third ventricle 20. Various methods are known for making this fenestration, including mechanical means, laser and ultrasonic vibration. Usually, fenestration 32 needs to be enlarged after initial formation to achieve a satisfactory size for the purpose of establishing a desired flow of CSF. Enlargement may be performed using a catheter or using an instrument designed for purposeful dilation of fenestrations. The catheter or the dilation instrument may be introduced through a working channel of the endoscope.

FIG. 3 shows a cross-sectional view of a portion of the floor of third ventricle 30 in which fenestration 32 has been established. Fenestration 32 is defined by perimeter edges 42 and 44. Where fenestration 32 has a circular shape, perimeter edges 42 and 44 are parts of the same continuous inner peripheral edge.

As previously discussed, up to 40% of ETV surgeries do not result in satisfactory resolution of hydrocephalus. A significant proportion of patients who fail to respond to ETV suffer from secondary re-closure of their ETV opening (fenestration 32) due to scarring and/or arachnoidal adhesion. This secondary occlusion of the fenestration can be avoided by use of the embodiments of the stent disclosed herein. The stent is typically an elongated device having resilient memory that allows it to expand from a radially compressed condition in which it is inserted into opening 32 to a radially expanded condition in which it is securely retained within opening 32.

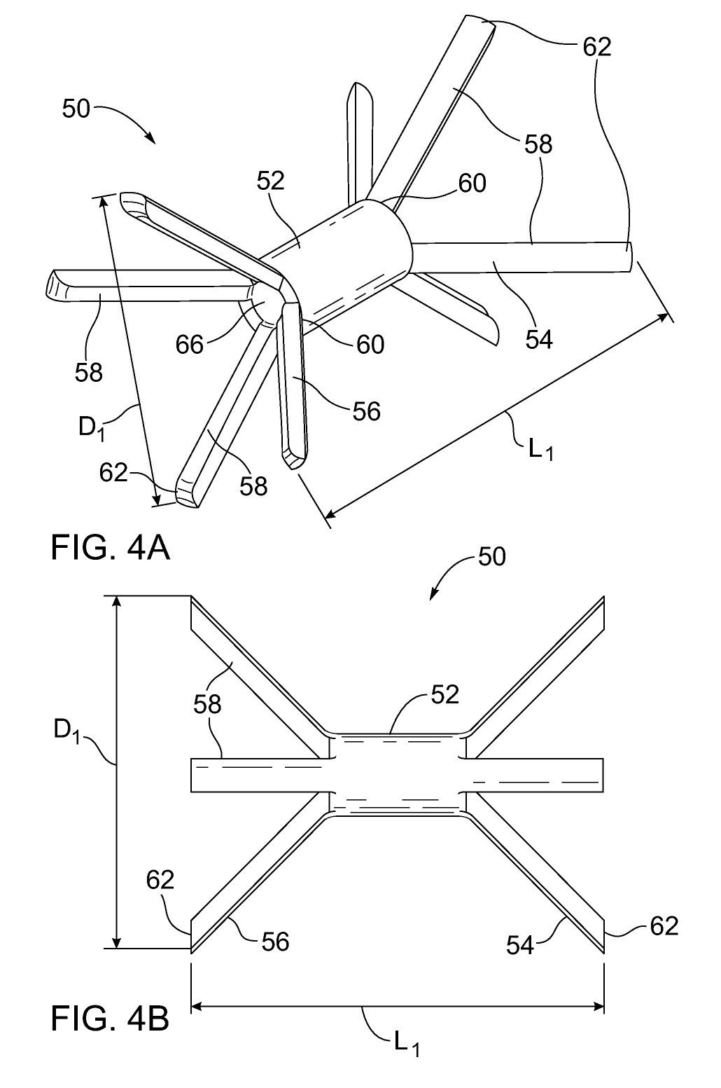

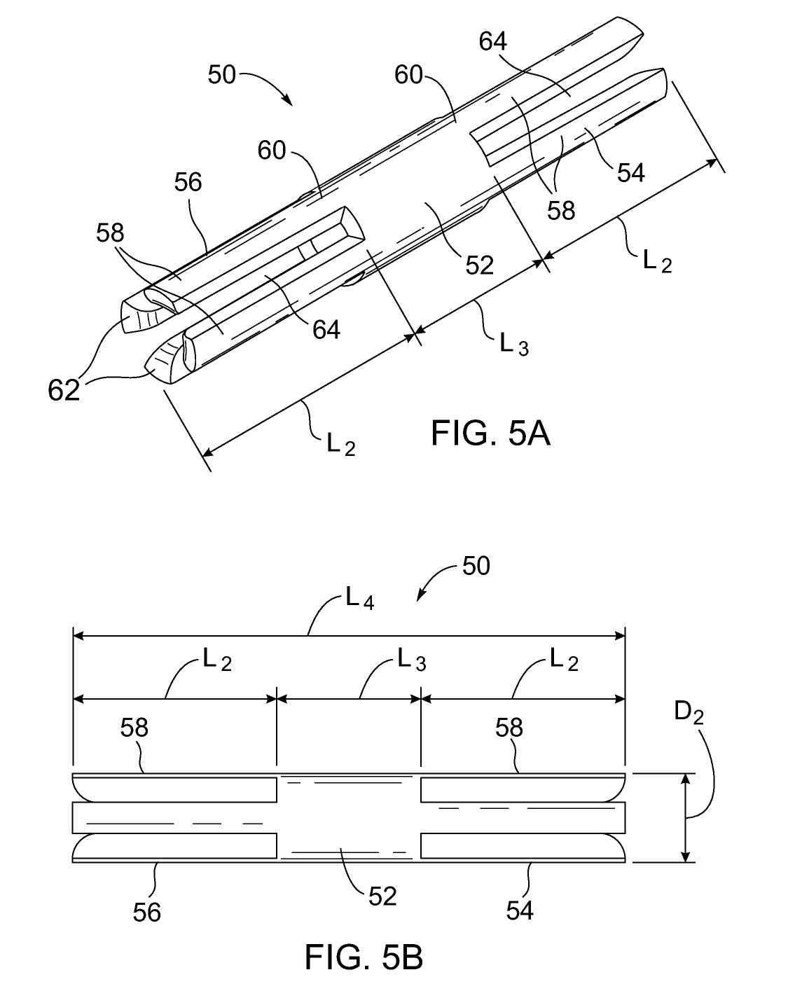

FIGS. 4 and 5 show a stent 50, according to one embodiment, that can be used to maintain the patency of an opening inside the body, such as fenestration 32. FIG. 4 shows the stent 50 in its expanded state and FIG. 5 shows the stent in its contracted, radially compressed state for delivery into the body. The stent 50 comprises an intermediate portion 52 and two relatively enlarged end portions 54, 56. Each end portion 54, 56 comprises a plurality circumferentially arrayed, axially elongated expandable fingers 58. Each finger 58 has a fixed end 60 hingedly connected to an adjacent end of the intermediate portion 52 and a free end 62. As best shown in FIG. 5, the fingers 58 can be separated from each other by longitudinally extending gaps 64. In other words, the fingers 58 in the illustrated embodiment are not connected to each other along their lengths and therefore can radially expand and contract relative to each other and the intermediate portion 52. The fingers 58 are normally biased outward to their expanded state. Thus, in the absence of a constraining force retaining the fingers 58 in the compressed state, the fingers expand radially outwardly from each other to the expanded state shown in FIG. 4.

The portion of each finger where the fixed end 60 is connected to the intermediate portion 52 can have a reduced thickness (relative to the remaining portion of the finger) so as to form a flexible hinge connecting the finger to the intermediate portion to facilitate radial movement of the finger relative to the intermediate portion.

The intermediate portion 52 has a central lumen, or passageway, 66 that allows fluids to flow through the stent. The intermediate portion 52 desirably is cylindrical as shown, although other shapes can be used. The intermediate portion 52 in the illustrated embodiment is a non-expandable component in that it is not compressed when loaded in a delivery sheath for delivery into a patient and does not expand when released from the delivery sheath. In other words, the intermediate portion 52 maintains its size and shape during delivery and deployment of the stent. Consequently, the intermediate portion 52 can be relatively rigid and/or substantially non-deformable. Alternatively, the intermediate portion 52 can be formed from a relatively soft, flexible and/or deformable material but can still be considered a non-expandable component because it need not be compressed to a smaller size for delivery into the patient. In other embodiments, however, the intermediate portion can comprise a radially expandable structure that can be compressed to a smaller diameter for delivery into the body and radially expands when deployed in the body, similar to a conventional coronary stent or any of the stent structures disclosed in U.S. Patent Application Publication No. 2007/0179426.

The intermediate portion 52, once implanted in an opening in the body, resists closure of the opening and functions as a fluid conduit to allow body fluids, such as cerebrospinal fluid or blood, to flow from one side of the opening to the other. For example, when implanted in a surgical fenestration formed in the floor of the third ventricle, cerebrospinal fluid can flow from the third ventricle to the subarachnoid space through the intermediate portion 52 of the stent. As shown, the intermediate portion 52 can comprise a tubular body having a substantially solid, cylindrical outer surface that extends between the opposite ends of the tubular body. The outer surface can be non-perforated as shown (i.e., not formed with any openings other than the openings at the opposing ends in communication with the central lumen) and substantially non-porous to body fluids, at least when initially implanted in the body. In particular embodiments, as described in detail below, the stent can be formed from a bioabsorbable material such that the stent dissolves within the body over a predetermined period of time. As the stent dissolves, the intermediate portion of the stent may become porous to body fluids. In alternative embodiments, the intermediate portion of the stent (whether formed from a bioabsorbable or a non-bioabsorbable material) can have a perforated structure, similar to a conventional coronary stent.

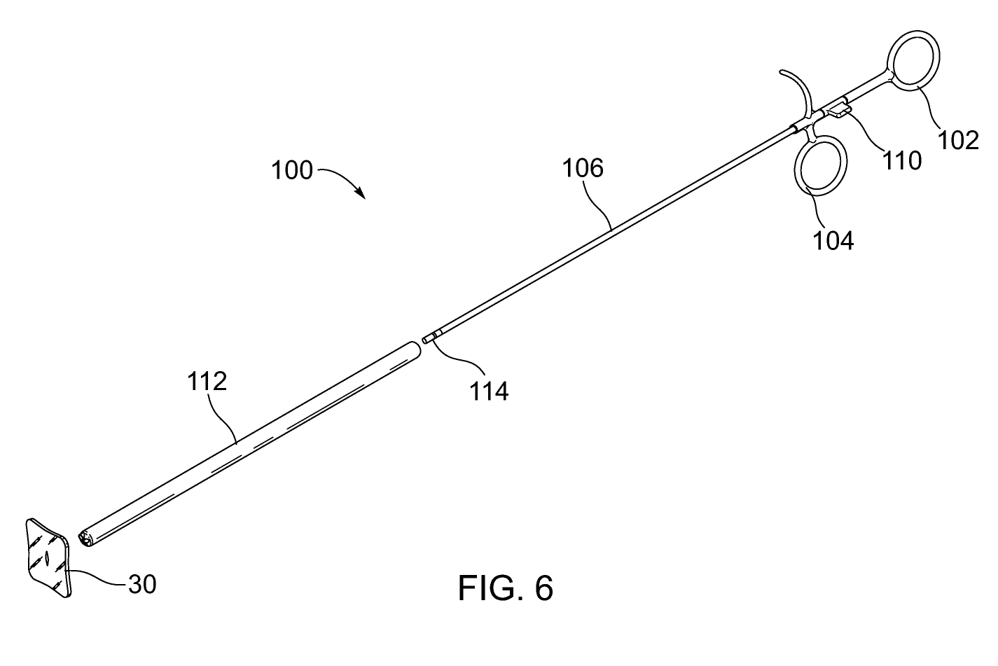

FIG. 6 is a perspective view of a delivery apparatus 100 (also referred to as a delivery catheter), according to one embodiment, that can be used to implant the stent 50 in a patient's body. The apparatus 100 comprises a proximal handle portion 102 and a distal handle portion 104. Extending from the distal handle portion 104 is an elongated sheath 106 having a central lumen. As best shown in FIG. 12, an elongated pusher element, or pusher rod, 108 is connected to the proximal handle portion 102 and extends through the distal handle portion 104 and the lumen of the sheath 106. The proximal handle portion 102 and the pusher element 108 are moveable longitudinally relative to the distal handle portion 104 and the sheath 106 to effect deployment of the stent 50, as further described below. A removable spacer element 110 (as best shown in FIG. 12) can be placed around the pusher element 108 between the handle portions 102, 104 to prevent inadvertent movement of the pusher element 108 relative to the sheath 106 in the distal direction, thereby preventing inadvertent deployment of the stent until it is positioned at the desired deployment position. In one embodiment, the spacer element 110 can be configured to form a snap fit connection around the pusher element 108 and can be removed from the pusher element by pulling or twisting the spacer element 110 relative to the pusher element.

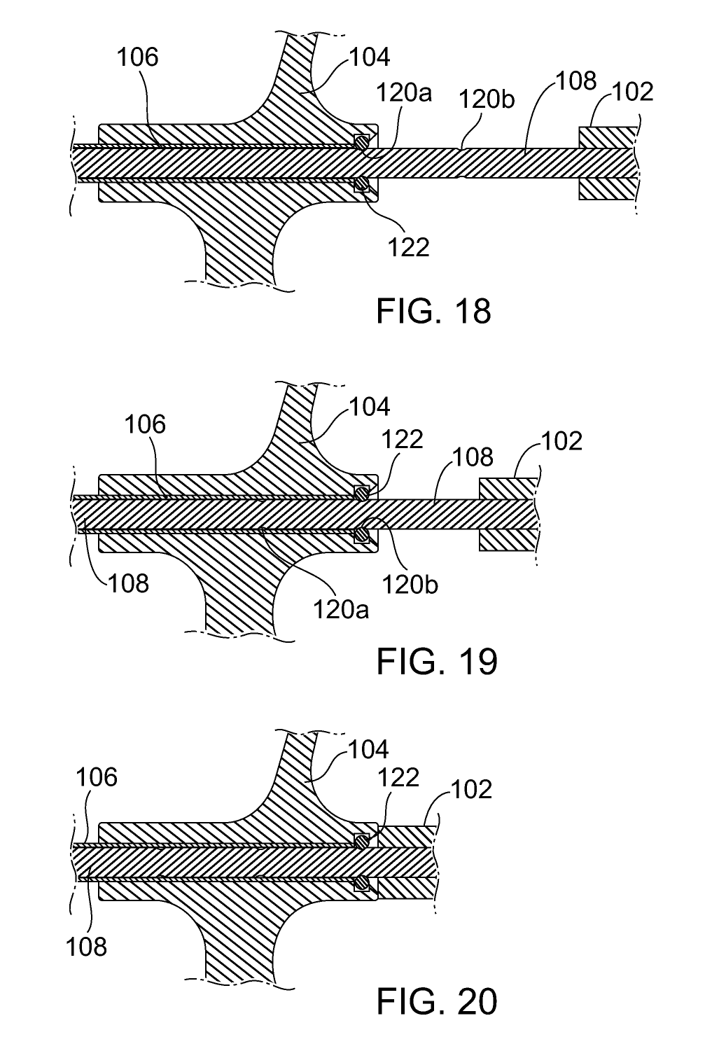

The pusher member 108 can be provided with a mechanism that provides for controlled advancement of the pusher member 108 relative to the sheath 106 for controlled deployment of the stent 50. For example, as best shown in FIGS. 18-20, the proximal end portion of the pusher member 108 (the end portion adjacent the handle portion 102) is formed with two axially spaced annular grooves 120a, 120b. The proximal end of the handle portion 104 contains an O-ring 122 that contacts the outer surface of the pusher member 108. As the pusher member is moved longitudinally relative to the sheath during stent deployment, the pusher member 108 slides relatively easily through the O-ring. However, when the O-ring 122 engages one of the grooves 120a, 120b on the pusher member, the sliding resistance of the pusher member relative to the sheath noticeably increases. This provides tactile feedback to the user corresponding to each stage of stent deployment for more controlled and accurate placement of the stent. The increase in sliding resistance caused by the O-ring engaging one of the grooves also helps prevent inadvertent movement of the pusher member relative to the sheath until the user applies sufficient manual force to the pusher member.

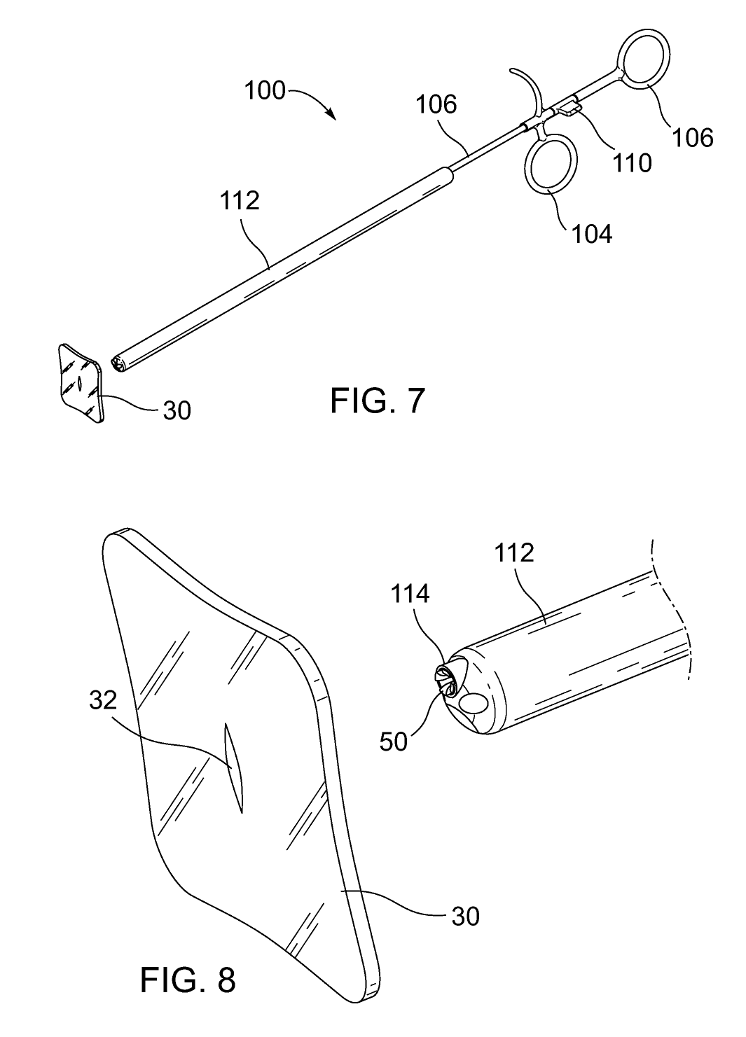

In use, the delivery apparatus 100 can be inserted into inserted into the body via a conventional trocar 112, which extends into the brain (or other operative site within the body) such that its distal end is spaced from fenestration 32. The trocar 112 serves as an endoscopic surgical port for accessing the operative site within the brain. FIGS. 7 and 8 show a portion of the floor 30 of the third ventricle and a fenestration 32 formed therein. The fenestration 32 can be formed in a conventional manner, such as by inserting a tool through the trocar 112 to access the floor 30. To introduce the stent 50 into the body, it is first inserted or loaded into a distal end portion 114 of the sheath 106. The sheath 106 retains the stent 50 in its compressed state while it is being introduced into the body. The distal end of the pusher element (not shown) abuts the proximal end of the stent 50 so that movement of the pusher element 108 relative to the sheath 106 in the distal direction is effective to push the stent out of the distal end of the sheath.

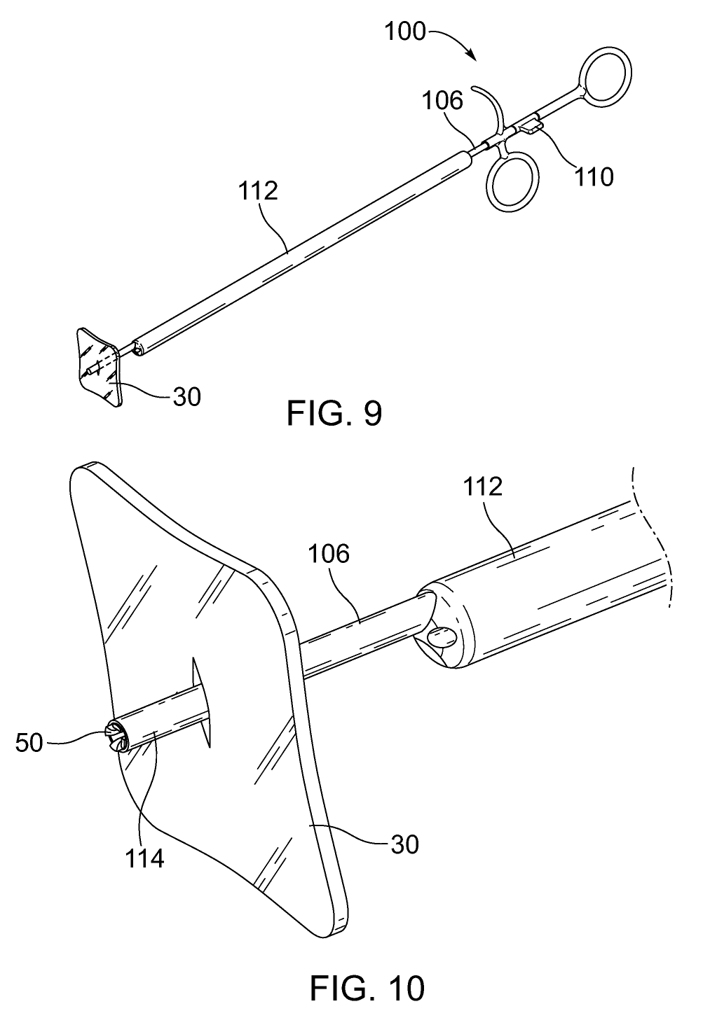

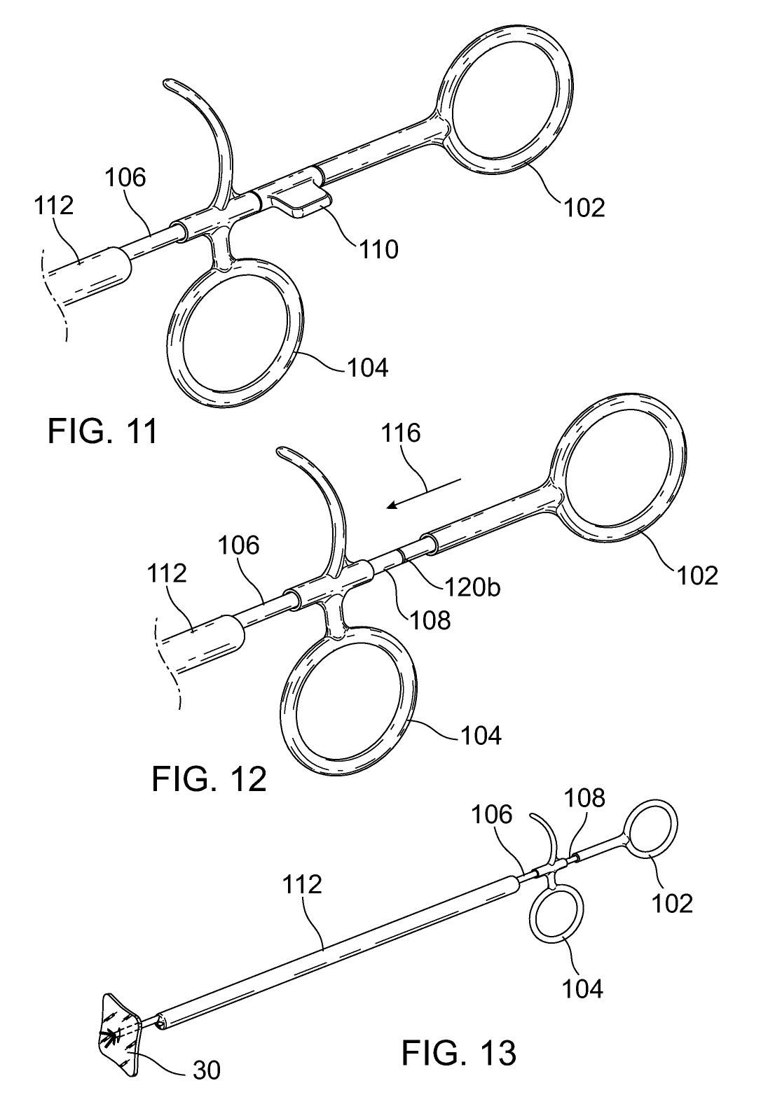

After the stent 50 is loaded in the delivery apparatus 100, it can be inserted through the trocar 112 until the distal end portion 114 of the sheath 106 extends through the fenestration 32, as depicted in FIGS. 9 and 10. The distal end portion 114 can be advanced relative to the fenestration 32 such that the stent 50 (in its compressed state in the sheath) is positioned on the opposite side of the floor 30 from the trocar (or at least the distal end portion 56 of the stent is positioned on the opposite side of the floor 30 from the trocar). At this stage, the spacer 110 can be removed from the pusher element 108. As best shown in FIG. 18, prior to stent deployment, the O-ring 122 is received in the distal groove 120a to further protect against inadvertent movement of the pusher member 108 until the user is ready to deploy the stent.

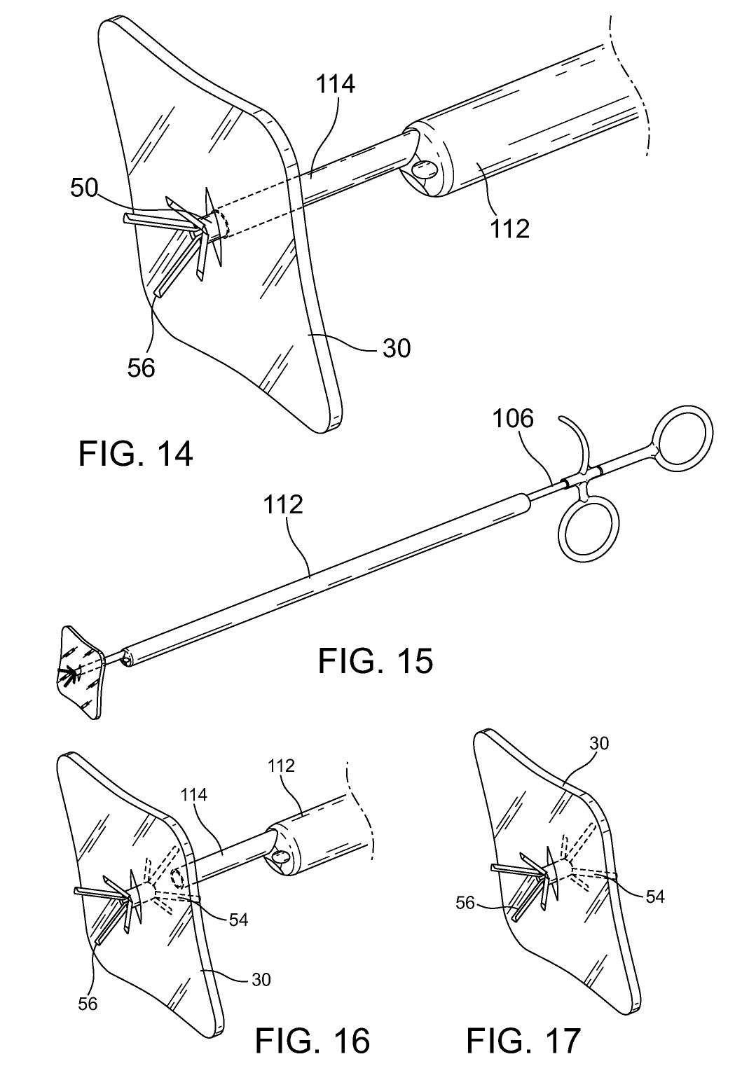

The distal end portion 56 of the stent 50 can then be deployed by holding the distal handle portion 104 stationary and pushing the proximal handle portion 102 in the distal direction, as indicated by arrow 116 (FIG. 12). The proximal handle portion 102 pushes the pusher element 108 relative to the sheath 106, which causes the distal end portion 56 of the stent to advance from the distal end of the sheath 106 and assume its expanded configuration shown in FIGS. 13 and 14. Alternatively, the stent can be deployed by holding the proximal handle portion 102 and the pusher element 108 stationary and retracting the distal handle portion 104 and the sheath 106 in the proximal direction relative to the proximal handle portion 102 and the pusher element 108. In any case, as the pusher element moves relative to the sheath (or vice versa), the O-ring 122 eventually engages the distal groove 120b when the distal portion 56 of the stent deploys from the sheath (FIG. 19). This provides tactile feedback to the user that the stent is partially deployed.

After the distal end portion 56 of the stent is deployed, the delivery apparatus 100 can be retracted slightly to bring the expanded end portion 56 into engagement with the adjacent surface of floor 30. This provides tactile feedback to the surgeon to help position the proximal end portion 54 on the opposite side of floor 30 from the distal end portion 56 before the proximal end portion 54 is deployed.

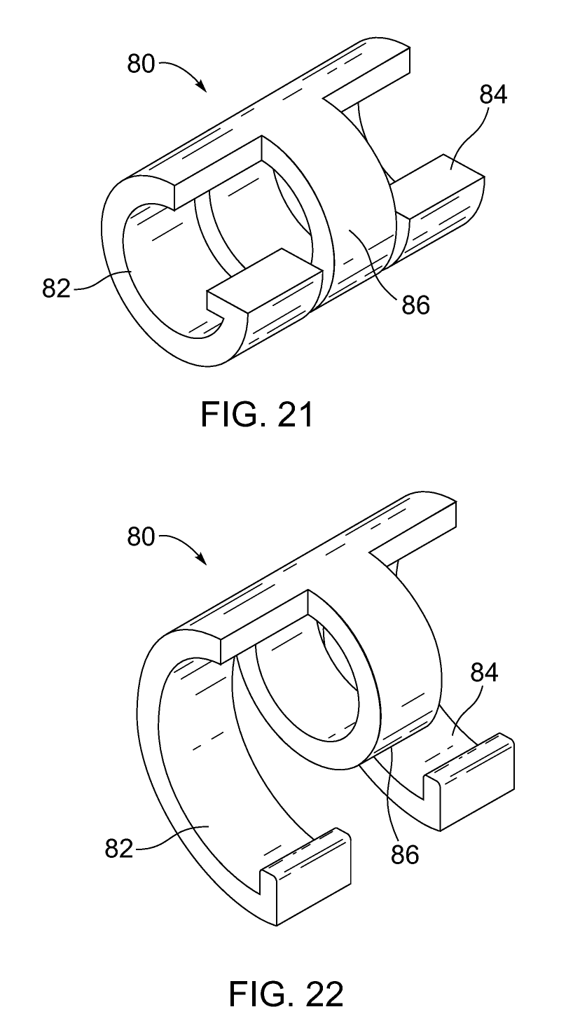

To deploy the proximal end portion 54 of the stent, the pusher element 108 is further pushed into the sheath 106 in the distal direction to push the remaining portion of the stent 50 outwardly from the sheath, allowing the proximal end portion 54 to expand to its expanded state, as depicted in FIGS. 15 and 16. FIG. 20 illustrates the position of the proximal handle portion 102 relative to the distal handle portion 104 after the stent is fully deployed from the sheath.

As shown in FIGS. 16 and 17, when the stent 50 is fully deployed, the end portions 54, 56 expand to a diameter greater than the diameter of the fenestration 32 to resist dislodgement of the stent in either direction out of the fenestration. For example, radially expanded ends 54, 56 can have a maximum diameter D₁ that is larger than that of intermediate portion 52 and also larger than the diameter of fenestration 32. In particular embodiments, referring to FIGS. 4A and 4B, diameter D₁ of enlarged ends 54, 56 desirably is at least ½ the length L₁ of the expanded stent 50, and more desirably at least ¾ the length L₁ of the expanded stent, and even more desirably at least the same as or greater than the length L₁. Length L₁ is measured along the longitudinal axis that is substantially perpendicular to the ends of the stent 50. The expanded diameter D₁ of the end portions 54, 56 is defined as the distance between the free ends 62 of two diametrically opposed fingers 58. In addition, the length L₂ of the end portions 54, 56 in their compressed state (which is also the same length of the fingers 58 in the illustrated embodiment) desirably is at least the same length or longer than the length L₃ of the intermediate portion 52.

The intermediate portion 52 can be configured to abut perimeter edges 42 and 44 of fenestration 32 to provide an anatomic barrier to closure of the opening due to inflammatory or other healing processes. However, since the stent 50 is hollow and both enlarged ends 54, 56 are open to fluid flow, retention of the stent 50 within fenestration 32 maintains patency of the fenestration 32.

Stent 50 is also preferably made of a bio-compatible material that degrades or otherwise spontaneously dissolves over a controlled or predetermined period of time that is sufficient to inhibit closure of fenestration 32. In many cases, natural inflammatory and healing processes, which initially tend to cause re-closure of the fenestrations, have by this point matured to form a stable and permanent scar tissue around the orifice, thus maintaining rather than occluding the opening. Once the stent has degraded after this period of time, the incidence of scarring or other closure of the opening is reduced.

In a particular example, that period of time is at least one month, for example one to six months. The time required for degrading the stent may be determined based on the observations of a typical interval during which a target opening may be subjected to undesired occlusion. For example, in ETV surgical procedures, the typical failure time during which the ventriculostomy opening may spontaneously close is several weeks. Accordingly, a suitable bioabsorbable material can be selected for making an ETV stent that degrades over several weeks after placement in the brain. For example, a material is chosen that is degraded by the continued flow of CSF through the stent in use. Gradual disappearance of the stent eliminates the necessity of surgical removal of the stent and also reduces the potential risk for infection or other failure that accompanies long term indwelling implants within the body. Furthermore, the bioabsorption time of the interfacial stent may be adjusted based on the selection of the material and/or the construction of the stent (e.g., selecting a mesh or generally solid construction for the stent.)

In particular embodiments of the stent, it can have the following dimensions:

TABLE 1
Dimensions of Stent in Compressed State

| (all dimensions in | Dimensions in |
|---|---|
| millimeters) | compressed state |
| Outer diameter D₂ | 1.5-2.5 |
| Overall length (L₄) | 4-15 |
| Length L₂ of end portions | 2-7 |
| 54, 56 and fingers 58 | |
| Length L₃ of intermediate | 2-8 |
| portion 52 | |
| Ratio of length L₄ to outer | 1.6:1-10:1 |
| diameter D₂ | |
| Ratio of finger length L₂ to | 0.25:1-3.5:1 |
| length of intermediate | |
| portion L₃ | |

TABLE 2
Dimensions of Stent in Expanded State

| (all dimensions in millimeters) | Dimensions in expanded state |
|---|---|
| Maximum diameter D₁ at ends of stent | 4.62-11.6 |
| Overall length L₁ | 5.8-12.8 |
| Ratio of D₁ to L₁ | .36-2.0 |

In a specific example, the stent 50 has the following dimensions: the outer diameter D₂ in the compressed state is about 1.8 mm; the overall length L₄ is about 11.48 mm; the length L₃ of the intermediate portion is about 3.0 mm; the length L₂ of a finger is about 4.24 mm; the ratio of L₄ to the outer diameter D₂ of the compressed stent is about 6.4:1; the ratio of L₂ to L₃ is about 1.4:1; the maximum diameter D₁ at the ends of the stent when expanded is about 7.8 mm; the maximum length L₁ of the expanded state is about 9.0 mm; and the ratio D₁ to L₁ is about 0.87:1.

Preferably, stent 50 is introduced into fenestration 32 during the same procedure in which the ventriculostomy fenestration is formed, such that stent 50 is introduced into fenestration 32 immediately after formation of that opening. After stent 50 has been deployed into ventriculostomy opening 32, the endoscopic tools used to introduce the stent into the opening are withdrawn from the body while leaving stent 50 in fenestration 32.

As shown in the above representative example, the present disclosure provides a method and device for inhibiting re-closure of openings in the human body, such as openings through biological interfaces that are designed to establish flow pathways. Re-closure is often caused by natural healing processes in the human body. Such healing processes are particularly effective in infants and young children, who indeed suffer a particularly high failure rate after anatomically successful ETV procedures. Infants and young children represent the majority of patients suffering from newly diagnosed obstructive hydrocephalus and thus would benefit most from the method and the stent of the present disclosure when applied in endoscopic third ventriculostomy.

The application of the method and the stent according to the present disclosure is not limited to ETV procedures. Examples of procedures in which maintenance of patency could be achieved in the disclosed fashion include a variety of cosmetic and therapeutic procedures. Patency of openings for body piercings could be assured, prior to introduction of a metal piercing, by placement of a biodegradable stent (which in this instance would not require a fluid passageway through it). Moreover, there are a number of therapeutic applications, such as maintaining patency of trabeculoplasty, trabeculotomy or sclerotomy openings in the eye for treatment of glaucoma; typanostomy openings in the eardrum for treatment of otitis media; tracheo-esphageal perforation for voice reconstruction after total laryngectomy; tracheostomy openings for establishing a patent airway bypass; openings created in endoscopic nasal and/or facial sinus surgery for maintaining mucous drainage pathways; openings for maintaining bronchopleural fistula for chronic drainage of pleural empyema and other disorders; and openings for the maintenance generally of other intentional permanent or semi-permanent fistulae in biological interfaces.

Although stent delivery has been described in connection with an endoscopic procedure, many other methods are known in the art that may be used to deliver the interfacial stent. In an endoscopic application as shown in the above representative example, existing endoscopic delivery systems may be readily adapted for delivery of the stent. For example, ETV surgery typically utilizes an endoscopic delivery port to deliver a catheter into the newly formed fenestration to enlarge the fenestration. The same endoscopic delivery port may be adapted for delivery of the interfacial stent. Although the stent can be conceivably deployed using a separate delivery port, sharing the same delivery port with the catheter simplifies the system.

In one embodiment, stent 50 is self-expandable, meaning that it expands autonomously when a compression or restraining force is removed, without requiring the application of external expansion forces (such as inflation of a balloon within the stent). One example of a self-expandable stent is a stent made of a polymer that has resilient memory, such that the stent expands in a controlled or predetermined fashion to assume a pre-configured shape, usually a shape that the stent had before it was subjected to compressive forces. Additional information about such polymers is provided in a later section of this specification.

Stent 50 also can be bioabsorbable, meaning that the stent will be dissolved or absorbed over time within the human body after a sufficient, usually predetermined period of time to maintain patency of the opening. In the present description, the terms “bioabsorbable”, “bioresorbable” and “biodegradable” have the same meaning and undistinguished from one another despite the awareness that some groups of individuals in the art may regard these terms to have different meanings.

FIGS. 18 and 19 show a stent 80, according to another embodiment. The stent 80 in the illustrated embodiment comprises a generally tubular body having a first end portion 82, a second end portion 84, and an intermediate portion 86. Each end portion 82, 84 comprises a cylindrical segment that is radially expandable between a compressed state (FIG. 18) and an expanded state (FIG. 19). When the stent is in the compressed state, it can be loaded into the sheath 106 of the delivery apparatus 100 for insertion into a patient's body. The end portions are self-expanding. In other words, when the stent is deployed from the sheath 106, the end portions automatically assume the expanded state shown in FIG. 19. The stent 80 can be implanted in the same manner as described above in connection with the stent 50.

### C. Stent Fabrication

As far as the manufacturing methods are concerned, several types of stents, including metal stents and polymer stents, may be suitable as the trans-interface stent of the present disclosure, with polymer stents being generally more preferable than metal stents.

Polymer Stents

Polymer stents include (but are not limited to) silicone, gelatin film, collagen film or matrix, polysaccharide matrices, and elastomer stents. Compared to metal stents, polymer stents are relatively newer products. One advantage that polymer stents have over metal stents is that they can be bioabsorbable/biodegradable. For this reason, polymer stents are more preferred for the applications disclosed herein.

An ideal stent may have the following characteristics (which are not essential requirements of the invention): (1) inexpensive to manufacture; (2) easy to deploy; (3) sufficiently rigid to resist radial forces; and (4) disappears after treatment without leaving behind harmful residue. Polymer devices that have this capability include resilient collagen materials, resilient gelatin films and biodegradable polymers such as polyesters, polyorthoesters, polyanhydrides, polyglycolic acid and poly(glycerol-sebacate) or PGS. For example, although less flexible, polyglycolic acid tubes provide results equivalent to silicone rubber but are absorbed in seven days and thereby obviate the need for any additional procedure to remove the stent. For applications in which it is desired that the stent have resilient memory, these biodegradable materials can be combined with other polymers that provide elastic recoil to a predetermined shape. A suitable biodegradable polymer available commercially is GELFILM®, an absorbable gelatin film made by Pharmacia & Upjohn (now a division of Pfizer).

Other suitable biodegradable polymers are discussed in U.S. Pat. No. 6,719,934, which patent is incorporated by reference to the extent that it discloses the polymers. These biodegradable polymers include polylactide bioabsorbable polymer filaments, helically wound and interwoven in a braided configuration to form a tube. Polylactide bioabsorbable polymer includes poly(alpha-hydroxy acid) such as poly-L-lactide (PLLA), poly-D-lactide (PDLA), polyglycolide (PGA), polydioxanone, polycaprolactone, polygluconate, polylactic acid-polyethylene oxide copolymers, modified cellulose, collagen, poly(hydroxybutyrate), polyanhydride, polyphosphoester, poly(amino acids), or related copolymers materials, each of which have a characteristic degradation rate in the body. For example, PGA and polydioxanone are relatively fast-bioabsorbing materials (weeks to months) and PLA and polycaprolactone are a relatively slow-bioabsorbing material (months to years). Another example of a suitable biodegradable polymer is trimethyl carbonate (TMC).

In addition, tyrosine-derived polycarbonate materials developed by Integra LifeSciences Holdings Corp. (Plainsboro, N.J.) may also be suitable for making the interfacial stents of the present disclosure. Another suitable example is bioresorbable, biocompatible and resilient bovine collagen materials developed by Integra LifeSciences Holdings Corp. Such collagen materials have been successfully used for various dental and surgical purposes, but a resilient form of such materials, either in filaments or sheets, may also be a good choice for fabricating the stents of the present disclosure.

A particular example of a biodegradable, self-expandable stent is the L-lactide-glycolic acid co-polymer with a molar ratio of 80:20 (SR-PLGA 80/20). This stent is sold under the product designation SpiroFlow (from Bionx Implants, Ltd., Tampere, Finland) and is disclosed in Laaksovirta et al., J Urol. 2003 August; 170(2 Pt 1):468-71. See also Chepurov et al., Urologiia. 2003 May-June; (3):44-50.

Other bioresorbable polymers under investigation by others may also be suitable. For example, a bioresorbable polymer stent incorporating natural polymers has been described by Bier and coworkers (Bier, J. D., et al., Journal of Interventional Cardiology, 1992. 5(3): p. 187-193.), where type I collagen was formed into a solid tube structure without slotted sides. Bioresorbable microporous intravascular stents were constructed by Ye and colleagues (Ye, Y.-W., et al., ASAIO Journal, 1996. 42: p. M823-M827. Ye, Y.-W., et al., Annals of Biomedical Engineering, 1998. 26: p. 398-408.). These stents were extremely porous, and a gradient could be produced from various surfaces of the stent.

As noted, a stent constructed of a bioabsorbable polymer provides certain advantages relative to metal stents such as natural decomposition into non-toxic chemical species over a period of time. Also, bioabsorbable polymeric stents may be manufactured at relatively low manufacturing costs since vacuum heat treatment and chemical cleaning commonly used in metal stent manufacturing are not required.

In addition, certain materials thought to be unsuitable for intraluminal stents used in vascular applications may be suitable for the stents disclosed herein. Intraluminal stents used in vascular applications have stringent requirements for materials to exhibit strong mechanical properties as structural support and desirable hemodynamics. Due to its distinctive application environment, interfacial stents may not require such stringent mechanical properties for the materials. For example, unlike the endovascular environment, an interfacial environment is less likely to exert high mechanical stress on the stent.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim, as our invention, all that comes within the scope and spirit of these claims.

I claim:

1. A stent comprising:
a non-expandable intermediate portion defining a lumen and having an outer diameter; and
first and second self-expandable end portions coupled to opposite ends of the intermediate portion, each of the first and second end portions comprising a plurality of circumferentially arrayed, axially elongated fingers that are radially expandable between a compressed state for delivery into a patient and an expanded state for deployment in the patient, each of the end portions having a maximum diameter in their expanded state that is greater than the outer diameter of the intermediate portion.

2. The stent of claim 1, wherein the stent is biodegradable.

3. The stent of claim 1, wherein the intermediate portion has a length and each of the end portions when compressed has a length that is equal to or greater than the length of the intermediate portion.

4. The stent of claim 1, wherein the intermediate portion has a length and the fingers have a length that is equal to or greater than the length of the intermediate portion.

5. The stent of claim 1, wherein the intermediate portion has an outer surface that is substantially non-porous to body fluids.

6. The stent of claim 1, wherein the fingers are completely separate from each other along their lengths.

7. The stent of claim 1, wherein the intermediate portion is cylindrical.

8. The stent of claim 1, wherein the intermediate portion has opposite ends and a substantially solid outer surface extending from one end to the other.

9. A method for maintaining patency of an opening inside the human body, comprising:
introducing a radially self-expanding stent into the body in a compressed state, the stent comprising a non-expandable intermediate portion defining a lumen and having an outer diameter, the stent further comprising first and second self-expandable end portions coupled to opposite ends of the intermediate portion, each of the first and second end portions comprising a plurality of circumferentially arrayed, axially elongated fingers;
positioning the first end portion on one side of the opening and allowing the fingers of the first end portion to radially expand to an expanded state; and
positioning the second end portion on an opposite side of the opening and allowing the fingers of the second end portion to radially expand to an expanded state such that the expanded fingers retain the stent within the opening.

10. The method of claim 9, wherein the stent is bioabsorbable, and degrades over time within the body after a sufficient period of time to maintain patency of the opening.

11. The method of claim 9, further comprising forming the opening by forming a surgical fenestration inside the human body.

12. The method of claim 11, wherein the surgical fenestration is formed in a wall of a ventricle of the brain to establish a path of cerebrospinal fluid flow from the ventricle to a sub-arachnoid space.

13. The method of claim 12, wherein the surgical fenestration is formed in a floor of the third ventricle.

14. The method of claim 11, wherein introducing the radially self-expanding stent into the body takes place substantially immediately after the fenestration has been artificially created.

15. The method of claim 9, wherein the stent comprises L-lactide-glycolic acid co-polymer, a biocompatible polymer, a biocompatible elastomer, a resilient collagen material, a polysaccharide matrix, or a bioabsorbable gelatin film.

16. The method of claim 9, wherein:
the stent is introduced into the body using a delivery device having a distal end portion comprising a sheath containing the stent in the compressed state, the delivery device further comprising a pusher member that is moveable axially relative to the sheath to deploy the stent from the distal end of the sheath; and
the method further comprises moving the pusher member relative to the sheath to cause the first end portion of the stent to advance from the sheath to allow the fingers of the first end portion to expand, and then subsequently further moving the pusher member relative to the sheath to cause the second end portion of the stent to advance from the sheath to allow the fingers of the second end portion to expand.

17. A method for treating hydrocephalus of a brain, comprising:
fenestrating the floor of the third ventricle of the brain of a patient to create an opening fluidly communicating between the third ventricle and a subarachnoid space;
introducing a radially self-expanding stent into the brain in a compressed state, the stent comprising a non-expandable intermediate portion defining a lumen and having an outer diameter, the stent further comprising first and second self-expandable end portions coupled to opposite ends of the intermediate portion, each of the first and second end portions comprising a plurality of circumferentially arrayed, axially elongated fingers;
positioning the first end portion on one side of the opening and allowing the fingers of the first end portion to radially expand to an expanded state; and
positioning the second end portion on an opposite side of the opening and allowing the fingers of the second end portion to radially expand to an expanded state such that the expanded fingers retain the stent within the opening.

18. The method of claim 17, wherein the stent is bioabsorbable.

19. The method of claim 17, wherein the intermediate portion has opposite ends and a non-perforated outer surface extending from one end to the other.
